(19) United States
(12) Patent Application Publication
(10) Pub. No.: US 2026/0198829 A1
(43) Pub. Date: Jul. 16, 2026

(54) DEVICES, SYSTEMS, AND METHODS FOR IMPLEMENTING SENSORS AND SENSOR ARRANGEMENTS VIA WEARABLE ARTICLES WITH FLEXIBLE CIRCUITS

(71) Applicant: Liquid Wire Inc., Portland, OR (US)

(72) Inventors: Mark William Ronay, Portland, OR (US); Jorge E. Carbo, JR., Portland, OR (US); Austin Michael Clarke, Tigard, OR (US); Michael Adventure Hopkins, Portland, OR (US); Cade Johnson, Canby, OR (US); Mark S. Kruskopf, Portland, OR (US); Jesse Michael Martinez, Milwaukie, OR (US); Katherine M. Nelson, Portland, OR (US); Trevor Antonio Rivera, Portland, OR (US); Michael Jasper Wallans, Portland, OR (US)

(73) Assignee: Liquid Wire Inc., Portland, OR (US)

(21) Appl. No.: 19/436,494

(22) Filed: Dec. 30, 2025

Related U.S. Application Data
(63) Continuation of application No. PCT/US24/36106, filed on Jun. 28, 2024.
(60) Provisional application No. 63/511,600, filed on Jun. 30, 2023.

(51) Int. Cl. A61B 5/256; A61B 5/00; A61B 5/296; A61B 5/389

(52) U.S. Cl. CPC A61B 5/256; A61B 5/296; A61B 5/389; A61B 5/7221; A61B 2562/164

(57) ABSTRACT

A wearable article is disclosed herein. The wearable article can include a flexible circuit that includes a first electrode including an outer surface of a first material and an inner surface of a second material, a substrate layer, a trace formed from a deformable conductor, and an encapsulation layer. The encapsulation layer can be configured to completely cover the trace and partially cover the first surface of the first electrode. Additionally, the wearable article can be communicably coupled to a processor and a memory configured to store instructions that, when executed by the processor, cause the processor to receive a first signal from the first electrode, receive a second signal from a second electrode, compare a first strength of the received first signal to a second strength of the received second signal, and deactivate the first electrode based on the comparison of the first strength and the second strength.

### CROSS-REFERENCE TO RELATED APPLICATIONS

The present application is a bypass continuation of and claims the benefit of priority from PCT Application No. PCT/US24/036106, filed 28 June 2024, which claims the benefit of priority from U.S. Provisional Patent Application No. 63/511,600, filed 30 June 2023, both of which are incorporated by reference in their entirety. All applications referenced herein are relevant to the subject matter disclosed herein and are hereby incorporated by reference in their entirety, regardless of the specific portion of the specification in which they are referenced.

### FIELD

The present disclosure is generally related to flexible and stretchable circuits and, more particularly, is directed to flexible and stretchable circuits that can be integrated into wearable articles.

### SUMMARY

The following summary is provided to facilitate an understanding of some of the innovative features unique to the aspects disclosed herein and is not intended to be a full description. A full appreciation of the various aspects can be gained by taking the entire specification, claims, and abstract as a whole.

Flexible wearable articles configured to be secured to a user are disclosed throughout the description. The wearable article includes a layer of material and a stretchable substrate coupled to the layer of material. The stretchable substrate comprises a circuit comprising an encapsulated deformable conductor and a sensor array. The wearable article further comprises a processor communicably coupled to the circuit and a memory. The memory is configured to store instructions that, when executed by the processor, cause the processor to, receive a signal from the sensor array, compare a strength of the received signal to a stored baseline threshold value, and ignore the received signal when the signal strength is less than the baseline threshold value. As used herein, the term “stretchable” can include a material that is able to revert to its original size and shape after being deformed, stretched, squeezed, or twisted.

These and other features and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Various features of the aspects described herein are set forth with particularity in the appended claims. The various aspects, however, both as to organization and methods of operation, together with advantages thereof, may be understood in accordance with the following description taken in conjunction with the accompanying drawings as follows:

FIG. 1 illustrates a plan view of a device configured to implement a sensor arrangement via flexible circuits, according to at least one non-limiting aspect of the present disclosure;

FIG. 2 illustrates a bottom view of the device of FIG. 1;

FIG. 3 illustrates a perspective view of the device of FIG. 1, secured about an appendage of a user, according to at least one non-limiting aspect of the present disclosure;

FIG. 4 illustrates another perspective view of the device of FIG. 1, according to at least one non-limiting aspect of the present disclosure;

FIG. 5 illustrates the perspective view of FIG. 4, sectioned about line 5-5;

FIG. 6 illustrates a plan view of another device configured to implement a sensor arrangement via flexible circuits, according to at least one non-limiting aspect of the present disclosure;

FIG. 7 illustrates an exploded, plan view of the device of FIG. 6;

FIG. 8 illustrates sensor of the device of FIG. 6;

FIGS. 9A and 9B illustrate sectioned views of a flexible trace deposition structure of the device of FIG. 6, according to several non-limiting aspects of the present disclosure;

FIG. 10 illustrates another sensor configured for use in a device with flexible circuits, according to at least one non-limiting aspect of the present disclosure;

FIG. 11A illustrates a sectioned view of a first portion of the sensor island of FIG. 9 showing an interface between sensors, conductive gel traces, and surrounding substrate layers in accordance with at least one aspect of the present disclosure.

FIG. 11B illustrates a sectioned view of a second portion of the sensor island of FIG. 9 showing an interface between conductive gel traces, an output device, and surrounding substrate layers in accordance with at least one aspect of the present disclosure.

FIG. 12 illustrates the sensor of FIG. 10 implemented on a device, according to at least one non-limiting aspect of the present disclosure;

FIG. 13 illustrates another sensor configured for use in a device with flexible circuits, according to at least one non-limiting aspect of the present disclosure;

FIG. 14 illustrates an exploded, perspective view of the sensor of FIG. 13;

FIG. 15 illustrates a flow chart of a method of selectively monitoring the output of a sensor array in accordance with at least one aspect of the present disclosure.

Corresponding reference characters indicate corresponding parts throughout the several views. The exemplifications set out herein illustrate various aspects of the invention, in one form, and such exemplifications are not to be construed as limiting the scope of the invention in any manner.

### DETAILED DESCRIPTION

Numerous specific details are set forth to provide a thorough understanding of the overall structure, function, manufacture, and use of the aspects as described in the disclosure and illustrated in the accompanying drawings. Well-known operations, components, and elements have not been described in detail so as not to obscure the aspects described in the specification. The reader will understand that the aspects described and illustrated herein are non-limiting examples, and thus it can be appreciated that the specific structural and functional details disclosed herein may be representative and illustrative. Variations and changes thereto may be made without departing from the scope of the claims. Furthermore, it is to be understood that such terms as “forward”, “rearward”, “left”, “right”, “upwardly”, “downwardly”, and the like are words of convenience and are not to be construed as limiting terms. In the following description, like reference characters designate like or corresponding parts throughout the several views of the drawings.

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves any and all copyrights disclosed herein.

The present disclosure is generally relevant to the disclosures of International Patent Application No. PCT/US2021/071374, titled WEARABLE ARTICLE WITH FLEXIBLE INDUCTIVE PRESSURE SENSOR, and filed September 3, 2021, International Patent Application No. PCT/US2023/062668, titled DEVICES, SYSTEMS, AND METHODS FOR CHARACTERIZING MOTIONS OF A USE VIA WEARABLE ARTICLES WITH FLEXIBLE CIRCUITS, and filed February 15, 2022, International Patent Application No. PCT/US2022/071012, titled DEVICES, SYSTEMS, AND METHODS TO MONITOR AND CHARACTERIZE THE MOTIONS OF A USER VIA FLEXIBLE CIRCUITS, and filed March 7, 2022, and U.S. Provisional Patent Application No. 63/412,867, titled DEVICES, SYSTEMS, AND METHODS FOR INTERACTING WITH & CALIBRATING A WEARABLE ARTICLE FEATURING CIRCUITS, and filed October 3, 2022, the disclosures of which are herein incorporated by reference in their entireties.

Wearable electronics continue to enhance our ability to monitor bodies in motion, such as a user’s physical parameters. However, the geometric configuration of a wearable article as well as the type and orientation of monitoring electronics will vary depending on the body part being monitored. Therefore, it would be beneficial for flexible electronic circuits to be incorporated into a wearable article for adaptable and reconfigurable musculoskeletal monitoring. Such circuits could expand and contract in accordance with the movement of a user, the securement of the wearable article to the user through wrapping, stretching, and/or twisting. Signals detected by a sensor array positioned on the flexible circuit can lead to one or more sensors being disabled and/or activated. Moreover, modular placement of the sensors within the sensor array could create more effective, personalized results from a general use product. Furthermore, the change in circuit geometry could lead to a subsequent change in electrical parameters generated across the circuit, which could be used to characterize a structural parameter or condition of the circuit, as desired.

While certain electronic components typically have some inherent flexibility, that flexibility is typically constrained both in the amount the components can flex, their resilience in flexing, and the number of times the electronic components can flex before the electronic components deteriorate or break. Consequently, the utility of such electronic components in various environments may be limited, either by reliability or longevity or by the ability to function at all.

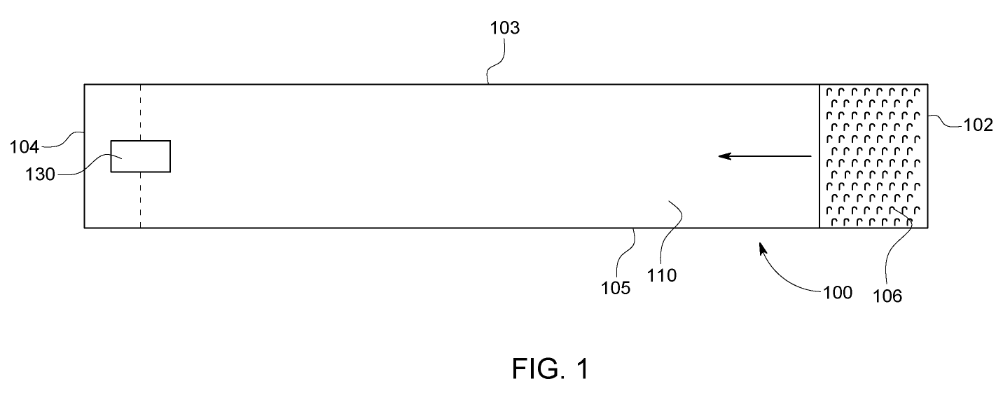

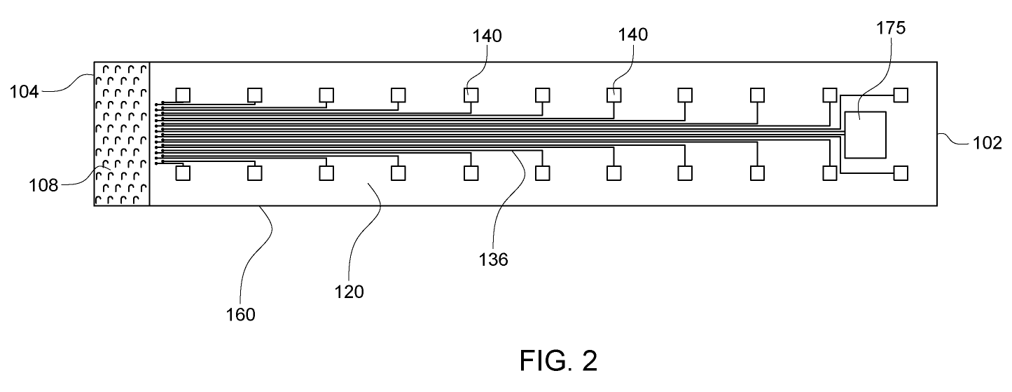

Referring now to FIG. 1, a plan view of a device configured to implement a sensor arrangement via flexible circuits is depicted according to at least one non-limiting aspect of the present disclosure. According to the non-limiting aspect of FIG. 1, the device can include a flexible wrap 100 capable of musculoskeletal monitoring through various sensors. The flexible wrap 100 is comprised of an elastic, stretchable material designed to be stretched, twisted, and/or otherwise secured to various portions of a user’s body including a chest, a knee, an elbow, a wrist, or any other portion of the user’s body of which monitoring is desired. Since many fibers, yarns, etc. used in fabrics or textiles and the like have some degree of stretchability, however small, it may be appreciated that “stretchable” as used in some embodiments to distinguish from “non-stretchable” refers to about 5% or more stretch with less than 0.5% growth using the ASTM D6614/D6614M-20 test protocol, and in the case of highly stretchable knit fabrics ASTM D2594 may be used. Other ASTM standards may be used when they are more appropriate, for example when the fabric is a thin polymer film, ASTM D882, and where the % stretch specified herein refers to the minimum strain of the film resulting in less than 0.5% elongation. If there is any uncertainty over what the best test method is for a given fabric type, or a fabric that comprises multiple layers of different fabric types (e.g., films, foams, wovens, knits, etc.), the test method of ASTM D6614 may be used and the same stretch and growth limits apply as the threshold value to determine the meaning of “stretchable”, even though this test standard may not have been designed specifically for this type of fabric. The flexibility of the wrap 100—and especially, flexible circuits integrated within the wrap 100—allows the wrap 100 to be utilized by users of all sizes and wrapped about any appendage or body part for monitoring via an arrangement of sensors 140, as will be depicted and described in further detail with reference to FIG. 2. As shown in FIG. 1, the wrap 100 can be generally rectangular in shape; however, any suitable shape, form factor, and/or size can be implemented. The boundaries of the wrap 100 are defined by a first end 102, a second end 104, a first edge 103, and a second edge 105. Accordingly, it shall be appreciated that the flexible wrap 100 can be wrapped about and secured to an appendage or body part of a user.

In various instances, the wrap is self-adherent. Stated another way, the material of the wrap clings and/or sticks together in such a manner that does not require any separate fastener to maintain the wrap in a desired orientation. In other instances, a fastening mechanism is provided to secure the wrap in a desired orientation. Exemplary fastening mechanisms include hook-and-loop fasteners, clips, and/or an adhesive. Such fastening mechanisms can be used individually or in combination with one another.

A first surface 110 of the wrap 100 is shown in FIG. 1, while an opposing, second surface 120 of the wrap 100 is shown in FIG. 2. The first surface 110 is optionally configured to face outward and away from a user’s skin when the user is wearing the flexible wrap 100. The second surface 120 is optionally configured to lay adjacent to and/or otherwise directly contact the user’s skin when the user is wearing the flexible wrap 100. The wrap 100 shown in FIGS. 1 and 2 has a fastening mechanism positioned on both the first and second surfaces 110, 120. More specifically, a textured loop region 106 is positioned on a first end 102 of the first surface 110 while a textured hook region 108 is positioned on a second end 104 of the second surface 120 of the wrap 100. In other instances, only the textured hook region 108 is necessary, as the hooks can releasably engage the material of an opposing portion of the wrap 100 to a degree sufficient to maintain the wrap in the desired orientation.

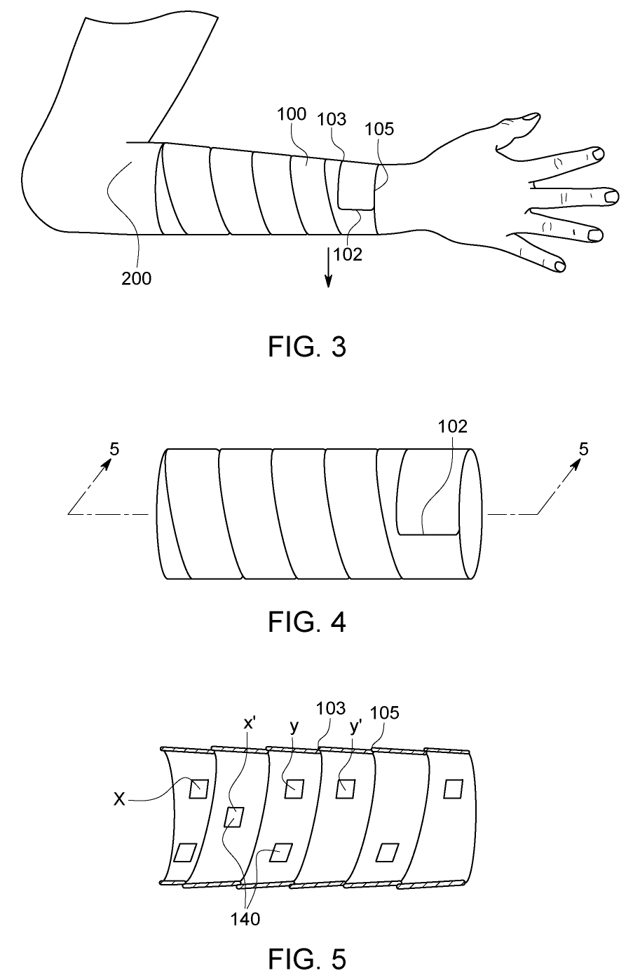

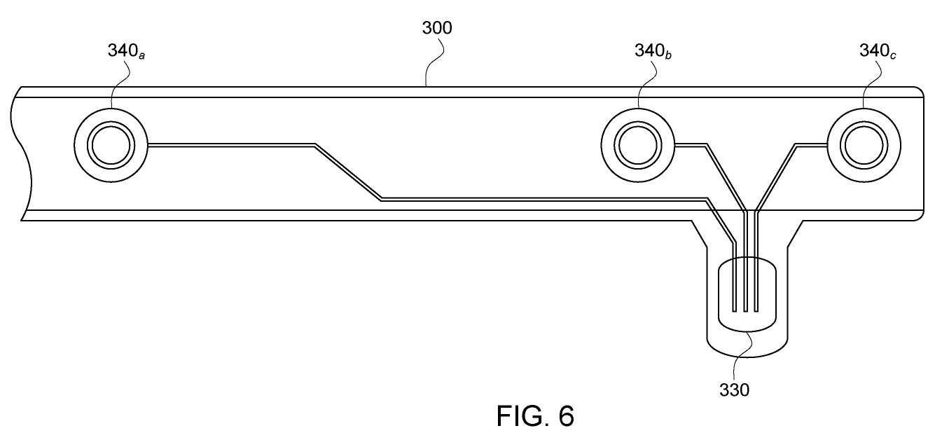

As shown in FIG. 3, the wrap 100 can be wound around a user’s forearm 200. FIGS. 4 and 5 show the wrap 100 in its wrapped configuration with the user’s forearm 200 removed. As shown in the partial cross-section taken along A-A depicted in FIG. 5, as the wrap extends around a desired portion of the user’s body, portions of the wrap may overlap one another to ensure the wrap has a tight, snug fit around the desired body portion, for example. As shown in FIGS. 3-5, as the wrap 100 extends around the user’s forearm 200, the first edge 103 repeatedly lays on top of and/or covers portions of the second edge 105. Furthermore, a user may overlap various portions of the wrap in an effort to achieve complete coverage of a body part, for example. In other instances, such as where the circumference of the body portion is larger, the wrap may not overlap itself.

More specifically, FIG. 5 depicts skin-facing surfaces of various sensors 140 positioned along the wrap 100. As described in greater detail herein, the wearable article is intended to be able to be secured to multiple parts of a wide size-range of user bodies. Due to such vast applicability, for example, when a user secures the wrap to his or her body, the sensors 140 may not always be in a desirable orientation and/ or in optimal contact with the user’s skin. For example, contact between the skin-facing surfaces of such sensors 140 and a user’s skin may be impacted and/or impeded by overlapping fabric of the wrap and/or a loosely secured wrap. In such instances, the signals emitted from and/or the information detected by such sensors are not representative of the body part intended to be monitored. In various instances, only one sensor is in optimal, desired contact with the user’s skin. As described in greater detail herein, the sensors of the sensor array are configured to be selectively switched on and off based on their emitted signal strengths relative to one another and/or their emitted signal strengths relative to a predetermined baseline value. For example, a first sensor x emits a first sensor signal having a first signal strength, a second sensor x’ emits a second sensor signal having a second signal strength, a third sensor y emits a third sensor signal having a third signal strength, and a fourth sensor y’ emits a fourth sensor signal having a fourth signal strength. A processor uses these emitted signals to selectively choose which sensors are appropriately monitoring the intended target. Based on this analysis, the processor can cause a power source to deactivate certain sensors, for example. The ability to deactivate certain mal-positioned sensors eliminates unnecessary noise from the sensor array. In instances where the sensors x, x’, y, and y’ are electrodes, the signal strengths may be analyzed in pairs, such as x-x’ and y-y’. In summary, one or more sensors 140 of the arrangement of sensors 140, for example, can be selectively activated by the user depending on the appendage or body part about which the flexible wrap. This can be accomplished via user inputs provided via a computing device (e.g., wearable computer, smart watch, smart phone, tablet, laptop, desktop computer, etc.) that is communicably coupled to a processing device, such as the processing device in the puck 130.

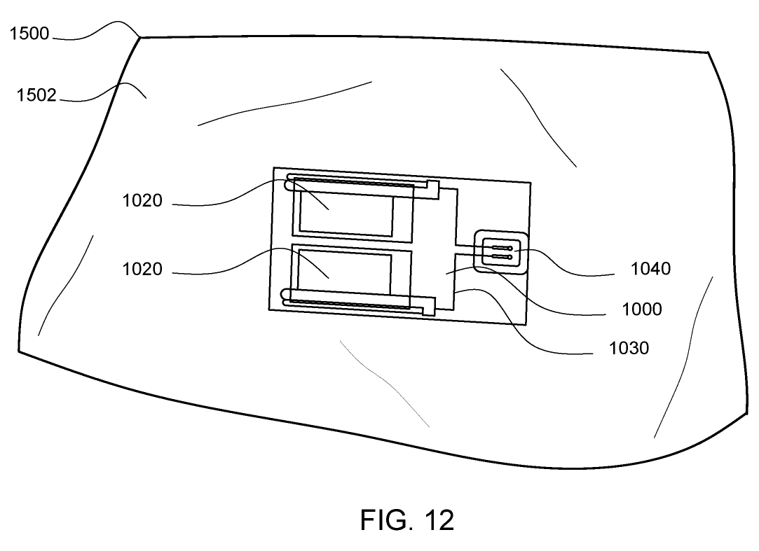

According to the non-limiting aspects of FIGS. 1-5, the device can include a flexible wrap 100 as the form factor for the disclosed wearable article. However, according to other non-limiting aspects, the flexible wrap can be geometrically configured to be wrapped around and secured about any appendage, including a back, leg, thigh, calf, finger, bicep, shoulder, neck, head, finger, and/or toe of the user, amongst other appendages. Moreover, although the flexible wrap of FIGS. 1-5 is substantially rectangular, according to other non-limiting aspects, the flexible wrap can include any number of alternate geometric configurations (e.g., triangular, oval, abstract, etc.) to facilitate a secure wrap about the desired appendage. Furthermore, according to some non- limiting aspects, the device can be configured as another wearable garment, such as a brace, sleeve, shirt, sock, headband, shorts, pants, and/or undergarments, amongst other garments to achieve a similar effect. However, it shall be appreciated that the flexible wrap 100 of FIGS. 1-5 can be wrapped about various appendages and thus, affords a flexibility beyond that of a garment particularly configured to be worn about a specific appendage or body part. For example, an alternate aspect is depicted in FIG. 12, wherein the device includes a flexible sleeve 1500. The sleeve 1500 can be worn independently, or the sleeve 1500 can be a part of a larger article of clothing such as a shirt, for example. While the wearable article is depicted in FIG. 12 as being a sleeve, exemplary form factors including a brace, a wrap, a shirt, a pair of pants, a pair of shorts, a sock, a shoe, a glove, and/or a hat, are all envisioned as suitable wearable articles.

The flexible wrap 100 monitors and/or collects real-time kinematic data through a sensor island 330 coupled thereto. The sensor island, or patch, 330 comprises a stretchable substrate 160 to which a sensor array is coupled thereto. As shown in FIG. 2, the arrangement (or array) of sensors 140 can include one or more sensors 140. In various instances, the sensors 140 are electromyography (EMG) electrodes used to detect one or more electrical signals emanating from skeletal tissue, however, according to other non-limiting aspects, other sensors are implemented (e.g., electrocardiogram sensors, hemodynamic/pressure monitoring sensors, optical sensors, chemical sensors, electromechanical sensors, hydration sensors, temperature sensors, etc.). The arrangement of sensors 140 can be arranged in an array such that each sensor is positioned in a particular location of the flexible wrap 100. According to the non-limiting aspect of FIG. 1, the detected EMG signals can reveal nerve dysfunction, muscle dysfunction, and/or problems with nerve-to-muscle signal transmission, for example. Motor neurons transmit electrical signals that cause muscles to contract. The EMG electrodes translate these signals into data that can be communicated to a puck 130 that includes electronic components, such as a processing device and/or a transceiver, and/or interpreted by a user and/or clinician. In other instances, the sensor 140 array includes at least one strain sensor 140, at least one pressure sensor, at least one temperature sensor, and/or some combination of any suitable sensor types.

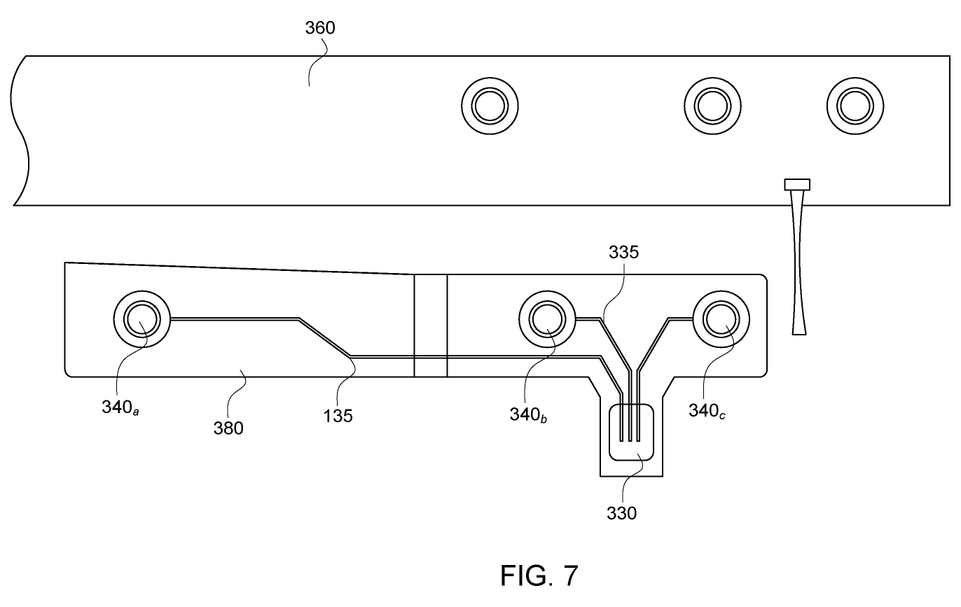

For example, the flexible wrap 100 may include a substrate and layup structure, which will be described in further detail with reference to FIGS. 6 and 7, that electrically couple the sensors 140 via traces 135 made from deformable conductors. The traces 135 can be integrated within a layup (including a substrate layer, a stencil layer, and/or an ecapsulation layer, etc.) and attached to a substrate 160 of the flexible wrap 100. The substrate 160, for example, can be composed of one or more flexible and stretchable materials, such as those disclosed by U.S. Patent Application No. 16/548,379 titled STRUCTURES WITH DEFORMABLE CONDUCTORS, which was filed on August 22, 2019, and granted as U.S. Patent No. 11,088,063 on August 10, 2021, the disclosure of which is hereby incorporated by reference in its entirety. The traces 135 deposited on the stretchable substrate 160 can include a deformable, conductive material, such as those disclosed in International Patent Application No. PCT/US2017/019762 titled LIQUID WIRE, which was filed on February 27, 2017 and published on September 8, 2017 as International Patent Publication No. WO2017/151523A1, the disclosure of which is hereby incorporated by reference in its entirety. For example, each trace 135 can include a variety of forms, such as a liquid, a paste, a gel, and/or a powder, amongst others that would enable the traces 135 to have a deformable (e.g., soft, flexible, stretchable, bendable, elastic, flowable viscoelastic, Newtonian, non-Newtonian, etc.) quality. According to some non-limiting aspects, the deformable, conductive materials can include an electroactive material, such as a deformable conductors produced from a conductive gel (e.g., a gallium indium alloy-based gel). The conductive gel can have a shear thinning composition and, according to some non-limiting aspects, can include a mixture of materials in a desired ratio. For example, according to one preferable non-limiting aspect, the conductive gel can include a weight percentage of a eutectic gallium alloy between 59.9% and 99.9% and a weight percentage of a gallium oxide between 0.1% and about 2.0%. Gallium alloy-based gels exhibit certain desirable characteristics that make it much more usable for patterning circuits on and/or within a variety of substrates and/or insulating materials such as those discussed in more detail herein. For example, the gel may have a viscosity, wettability, and/or adherability to a variety of rigid, flexible and/or stretchable films and structures. Of course, the present disclosure contemplates other non-limiting aspects, featuring traces 135 of varying forms and/or compositions to achieve the benefits disclosed herein. The deformable nature of the traces 135 allows for the wrap 100 to be stretched and/or twisted by a user without losing connectivity between the one or more electrodes 140 and the puck 130. Such deformability of the traces 135 further allows the wrap 100 to be effectively worn by users of all sizes and secured to numerous body parts.

The traces 135 may be deposited on the stretchable substrate 160 in any suitable pattern and/or quantity to provide for modularity of the wearable article. Stated another way, the traces 135 can be present in an adequate quantity and position to enable a user to selectively orient electrodes, IMUs, and/or other suitable sensors along the substrate 160.

The network of conductive gel traces 135 can simultaneously serve as a strain sensor, the individual electrical characteristics of which translates to a relative length or other orientation of the trace. By combining the electrical characteristics, e.g., by triangulating or other mathematical process, the relative location of various points on a two- dimensional surface may be determined. By measuring such electrical characteristics repeatedly over time, the motion of the points may be determined, providing for the capacity for real-time motion capture of the points on the strain sensor. By scaling the network of traces and/or including discrete strain sensors that may overlay certain features of an object, e.g., in the case of a body member of a mammalian or other creature, over joints or muscles and/or muscle groups, the motion of the object may be inferred in real-time by correlating the measured values output by the sensors to a model (such as a mathematical model, tabular empirical values, a virtual representation, an algorithm, or otherwise) of the object’s structure. The network of conductive gel traces 135 or discrete strain sensors can be coupled to the substrate 160 and/or the puck 130.

According to other non-limiting aspects, the traces 135 can be alternately and/or additionally configured as a bus circuit for transporting electrical power to various components, and/or an antenna circuit for transmissions, amongst other circuit types.

Alternatively and/or additionally, the one or more traces 135 can undergo a fluid-type strain and/or shear within the trace 135. The gel composition of the traces 135 allows for a shear thinning material that flows readily when it undergoes a shearing-type stress. Thus, when the substrate 160 is relaxed and returns to pre-strained state, the trace 135 will return to a static, or sedimentary, viscosity. In other words, the one or more traces 135 shall flow as the substrate 160 is deformed and thus, will not preclude the circuits disclosed herein from undergoing the requisite transition between a first (e.g., non-stretched) state to a second (e.g., stretched and/or twisted) state while preserving electrical communication throughout the circuit in either the first or second state. In this regard, the one or more traces 135 can include a deformable conductor that is distinguished from conventional conductors, such as copper wires, which would prevent the transition between the first and second states, or whose rigidity might break the electrical communication throughout the circuit during said transitions.

Such EMG electrodes can be surface electrodes used to provide a non-invasive technique for measurement and detection of the EMG signal. Surface EMG electrodes form a chemical equilibrium between the detecting surface 142 and the skin of the body through electrolytic conduction, allowing current can flow into the electrode. As such, the detecting surface 142 of the EMG electrodes is configured to face and be in contact with the skin of a user and may be exposed on the second surface 120, whereas if the sensor array included, e.g., strain sensors, such sensors may be exposed on any of the first surface 110, the second surface 120 or may be embedded between or within any layers of the wrap 100 and not be outwardly exposed on any of the surfaces 110, 120.

In various instances, the sensor array comprises one or more inertial measurement units (IMUs). An inertial measurement unit can be positioned on the first end 102 of the second side 120 of the wearable article, on the second end 104 of the second side 120 of the wearable article, and/or on any suitable position of the second side 120 of the wearable article. The one or more IMUs can further be arranged in any desired orientation and/or pattern on the wearable article. The substrate 160 may only have one or more IMU affixed thereto. Stated another way, in some instances where one or more IMU is affixed to the substrate 160, the substrate 160 does not have any electrodes 140 supported thereon.

The puck 130, on its own or hosted as part of an auxiliary component such as on a flex printed circuit board (“PCB”), can be coupled to the stretchable substrate 160 and configured to correlate varying electrical signals to an associated parameter. The puck 130 may include a processor, an analog to digital converter (ADC), an electronic data storage, and/or a system input/output. The processor may be a conventional processor, microprocessor, controller, microcontroller, or any suitable processing or controlling device. The processor may receive the output from the sensor array. The electronic data storage may be any one or more of a volatile or non-volatile electronic data storage, such as memory, hard drive, cache, or the like. The processor can be communicably coupled to the electronic data storage configured to store instructions that, when executed by the processor, cause the processor to characterize the signals received from the sensor array as described in greater detail herein. The ADC may convert analog signals (e.g., signals from the sensor array) to digital signals for interpretation by the processor. The system input/output may be provided to communicate outside of the wearable article.

In various instances, the puck 130 can include a rechargeable power source (e.g., a lithium-ion battery, a capacitor, etc.) configured to deliver an electrical current to the circuit and/or a port (e.g., a universal serial bus (USB) port) configured to directly deliver an electrical current to the circuit and/or charge the power source, itself. According to other non-limiting aspects, the puck can include one or more electronic components including a power source, such as a battery and/or a charger. The charger, for example, can include a universal serial bus (“USB”) port configured to convey electrical power and/or data to the sensor array from an external source. For example, the sensor array can be configured for such conveyance via a USB-A, USB-B, or USB-C protocol, although other means for power and/or data conveyance are contemplated by the present disclosure. According to other non-limiting aspects, the sensor array can include a wireless charging circuit and/or a wireless transmitter and/or receiver configured to wirelessly obtain power and/or data from external sources. Additionally, via one or more electronic components, it shall be appreciated that data can be transmitted to and from the puck 130. For example, according to some non-limiting aspects, one or more electronic components can be used to transmit a firmware update to a memory of the wrap, for execution by a microprocessor. Alternately, the one or more electronic components can include a memory configured to store data generated by the sensor array for subsequent use and processing.

One or more of the components associated with the puck 130 can be hosted on the wearable article itself. Alternatively, the puck 130 can be an output device, such as a USB connector, a pin-out connector, or an intermediate contact to selectively interface with a secondary circuit, for example.

The stretchable substrate 160 further comprises a network of conductive gel traces 135 extending between electrical contacts, the sensors (including electrodes, IMUs, and/or suitable combinations thereof) 140, the sensor island 330 (which can include an IMU), and/or the puck 130. The traces 135 deposited on the stretchable substrate 160 can include a deformable, conductive material, such as those disclosed in International Patent Application No. PCT/US2017/019762 titled LIQUID WIRE, which was filed on February 27, 2017 and published on September 8, 2017 as International Patent Publication No. WO2017/151523A1, the disclosure of which is hereby incorporated by reference in its entirety. For example, each trace 135 can include a variety of forms, such as a liquid, a paste, a gel, and/or a powder, amongst others that would enable the traces 135 to have a deformable (e.g., soft, flexible, stretchable, bendable, elastic, flowable viscoelastic, Newtonian, non-Newtonian, etc.) quality. According to some non-limiting aspects, the deformable, conductive materials can include an electroactive material, such as a deformable conductors produced from a conductive gel (e.g., a gallium indium alloy-based gel). The conductive gel can have a shear thinning composition and, according to some non-limiting aspects, can include a mixture of materials in a desired ratio. For example, according to one preferable non-limiting aspect, the conductive gel can include a weight percentage of a eutectic gallium alloy between 59.9% and 99.9% and a weight percentage of a gallium oxide between 0.1% and about 2.0%. Gallium alloy-based gels exhibit certain desirable characteristics that make it much more usable for patterning circuits on and/or within a variety of substrates and/or insulating materials such as those discussed in more detail herein. For example, the gel may have a viscosity, wettability, and/or adherability to a variety of rigid, flexible and/or stretchable films and structures. Of course, the present disclosure contemplates other non-limiting aspects, featuring traces 135 of varying forms and/or compositions to achieve the benefits disclosed herein. The deformable nature of the traces 135 allows for the wrap 100 to be stretched and/or twisted by a user without losing connectivity between the one or more electrodes 140 and the puck 130. Such deformability of the traces 135 further allows the wrap 100 to be effectively worn by users of all sizes and secured to numerous body parts.

The traces 135 may be deposited on the stretchable substrate 160 in any suitable pattern and/or quantity to provide for modularity of the wearable article. Stated another way, the traces 135 can be present in an adequate quantity and position to enable a user to selectively orient electrodes, IMUs, and/or other suitable sensors along the substrate 160.

The network of conductive gel traces 135 can simultaneously serve as a strain sensor, the individual electrical characteristics of which translates to a relative length or other orientation of the trace. By combining the electrical characteristics, e.g., by triangulating or other mathematical process, the relative location of various points on a two- dimensional surface may be determined. By measuring such electrical characteristics repeatedly over time, the motion of the points may be determined, providing for the capacity for real-time motion capture of the points on the strain sensor. By scaling the network of traces and/or including discrete strain sensors that may overlay certain features of an object, e.g., in the case of a body member of a mammalian or other creature, over joints or muscles and/or muscle groups, the motion of the object may be inferred in real-time by correlating the measured values output by the sensors to a model (such as a mathematical model, tabular empirical values, a virtual representation, an algorithm, or otherwise) of the object’s structure. The network of conductive gel traces 135 or discrete strain sensors can be coupled to the substrate 160 and/or the puck 130.

According to other non-limiting aspects, the traces 135 can be alternately and/or additionally configured as a bus circuit for transporting electrical power to various components, and/or an antenna circuit for transmissions, amongst other circuit types.

Alternatively and/or additionally, the one or more traces 135 can undergo a fluid-type strain and/or shear within the trace 135. The gel composition of the traces 135 allows for a shear thinning material that flows readily when it undergoes a shearing-type stress. Thus, when the substrate 160 is relaxed and returns to pre-strained state, the trace 135 will return to a static, or sedimentary, viscosity. In other words, the one or more traces 135 shall flow as the substrate 160 is deformed and thus, will not preclude the circuits disclosed herein from undergoing the requisite transition between a first (e.g., non-stretched) state to a second (e.g., stretched and/or twisted) state while preserving electrical communication throughout the circuit in either the first or second state. In this regard, the one or more traces 135 can include a deformable conductor that is distinguished from conventional conductors, such as copper wires, which would prevent the transition between the first and second states, or whose rigidity might break the electrical communication throughout the circuit during said transitions.

The substrate 160 may be composed of one or more flexible and stretchable materials, such as those disclosed by U.S. Patent Application No. 16/548,379 titled STRUCTURES WITH DEFORMABLE CONDUCTORS, which was filed on August 22, 2019, and granted as U.S. Patent No. 11,088,063 on August 10, 2021, the disclosure of which is hereby incorporated by reference in its entirety. Specifically, the substrate 160 can be fabricated from a flexible or stretchable material such as a natural rubber, a synthetic rubber, a flexible plastic, a silicone-based material (e.g., polydimethylsiloxane (“PDMS”), thermoplastic polyurethane (“TPU”), ethylene propylene dieneterpolymer (“EPDM”), neoprene, polyethylene terephthalate (“PET”), etc.), a flexible composite material, and/or a naturally flexible material, such as a leather, for example. For example, the substrate can be fabricated from a resilient, albeit stretchable TPU, such as Lubrizol^{®} Estane^{®} 58000 series (e.g., 58238), amongst others. Alternatively, the substrate 160 can be formed from a flexible, though comparatively more rigid material, such as Lubrizol^{®} Estane^{®} S375D, amongst others. The substrate 160 can include a single layer configured to accommodate the traces 135 directly printed thereon. In other instances, the substrate can include a multi-layer construction—including a substrate layer, a stencil-layer, and an encapsulation layer—in other non-limiting aspects, the substrate 160 can include a two-layer construction (e.g., substrate layer, encapsulation layer, etc.). According to some non-limiting aspects, the substrate 160 can be made with any suitable compressible material (e.g., flexible, rigid with some flexibility, or deformable). For example, the substrate 160 may be a rigid plate that may be bent/deformed/compressed in response to a motion of a user (e.g., bending a joint) that deforms it. According to other non-limiting aspects, the substrate 160 can include viscoelastic properties such that the substrate 160 can absorb vibrations. The substrate 160 can be formed from a viscoelastic film, similar to those used in a 3M^{TM} VHB^{TM} viscoelastic tape, for example.

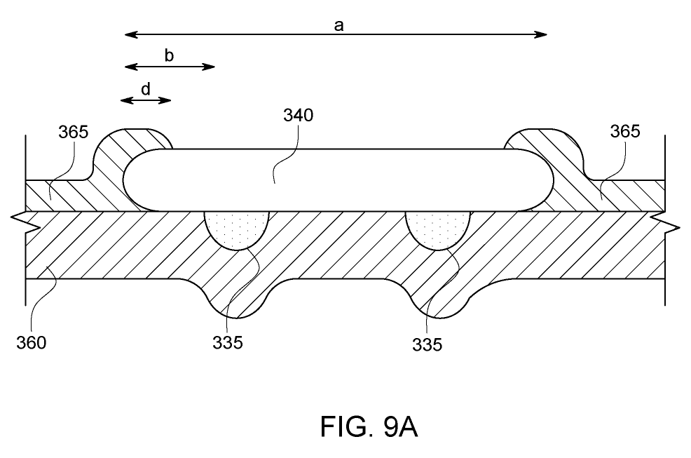

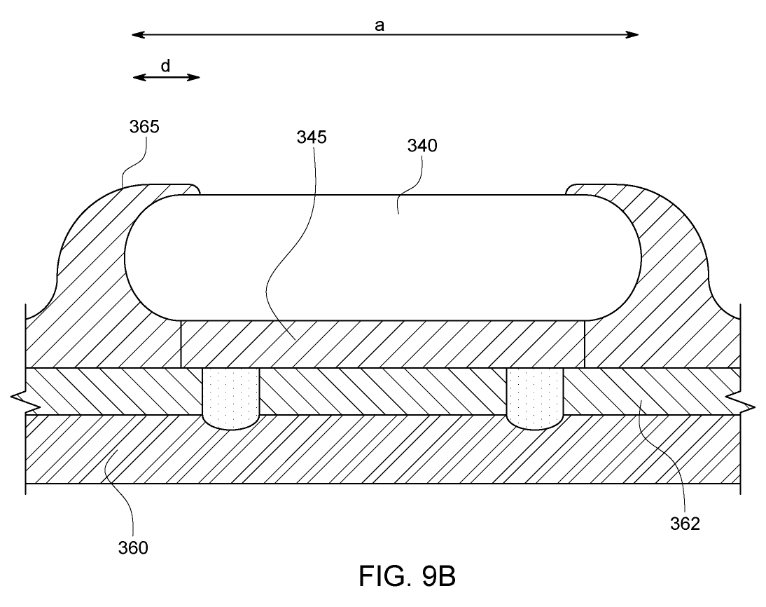

Referring now to FIG. 6, a plan view of another device configured to implement a sensor arrangement via flexible circuits is depicted according to at least one non-limiting aspect of the present disclosure. According to the non-limiting aspect of FIG. 6, the device can include a flexible wrap 300 capable of musculoskeletal monitoring through an alternate arrangement of sensors 340_{a-c} connected via one or more deformable traces 306_{a-c} configured similar to the flexible wrap 100 of FIG. 1. For example, the flexible wrap 300 can include an elastic, stretchable material designed to be stretched, twisted, and/or otherwise secured to various portions of a user’s body including a chest, a knee, an elbow, a wrist, or any other portion of the user’s body of which monitoring is desired. According to the non-limiting aspect of FIG. 6, the sensors 340_{a-c} can include EMG sensors; however, according to other non-limiting aspects, any of the aforementioned sensors can be implemented to achieve a similar effect. Additionally, according to some non-limiting aspects, the one or more sensors 340_{a-c} can be selectively activated in accordance with user preference and/or to accommodate for a body part or appendage the flexible wrap 300 is secured about. However, the integration of the sensors 340_{a-c} into the flexible wrap 300 is of particular interest, as will be described in further detail with reference to FIGS. 9A and 9B.

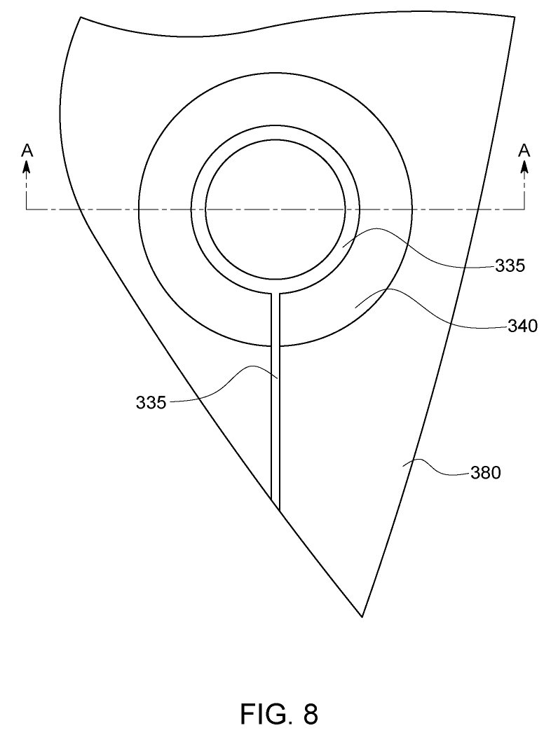

Referring now to FIG. 7, an exploded, plan view of the flexible wrap 300 of FIG. 6 is depicted in accordance with at least one non-limiting aspect of the present disclosure. According to the non-limiting aspect of FIG. 7, the flexible wrap 300 can include a substrate layer 360 and a layup 380 from which a flexible circuit that includes the traces 335, the sensors 340_{a-c}, and an electronic component 330 (such as a sensor island, or a puck) of the flexible wrap 300. FIG. 8 provides a more detailed overview of the sensor 340 of the flexible wrap 300, according to at least one non-limiting aspect of the present disclosure. For example, the sensor 340 can represent any of the sensors 340_{a-c} depicted in FIGS. 6 and 7 and can include an EMG electrode that is electrically coupled to a trace 335 of the layup 380 of the flexible circuit. The present disclosure will not provide several details regarding the specific integration of the sensor 340 and trace 335 within the layup 380 structure of the flexible circuit.

For example, referring now to FIG. 9A, a sectioned view of a flexible trace 335 deposition structure of the flexible wrap 300 of FIG. 6 is depicted according to at least one non-limiting aspect of the present disclosure. Specifically, the section of FIG. 9A was taken along lines A-A in FIG. 8. The various layers are presented for illustration and not limitation, and it is to be recognized and understood that any of a variety of additional or alternative layers may be incorporated into the laminate structure as desired. As depicted in FIG. 9A, conductive gel traces 335 are positioned on a base substrate layer 360. An electrode 340 is encapsulated against the base substrate layer 360 and the conductive gel traces 335 by at least one encapsulation layer 365. The encapsulation layer 365 covers enough of the electrode 340 to maintain the electrode’s desired position and/or orientation without impeding the surface area of the electrode’s skin-facing surface. Stated another way, the encapsulation layer 365 serves to adequately secure the electrode 340 against the base substrate layer 360 while also maximizing the electrode’s skin-facing surface. As shown in FIG. 9A, the electrode 340 comprises a first width “a.” In order to maintain sufficient contact with the conductive traces 335, for example, the electrode 340 is positioned on the base substrate layer 360 in such a manner that the conductive traces 335 are a distance “b” inside of an outer edge, or boundary, of the electrode 340. Furthermore, the encapsulation layer 365 is configured to overlap the electrode 340 by a distance “d.” In various instances, the magnitude of “b” and “d” are the same. In other instances, the magnitude of “b” is different than the magnitude of “d.” For example, the magnitude of “b” can be greater than the magnitude of “d.” Generally, the electrode 340 is geometrically configured and placed on the base substrate layer 360 relative to the conductive gel traces 335 and encapsulated in a manner that creates an acceptable aspect ratio and ensure proper electrical contact. The amount of encapsulation overlap must be sufficient to secure the electrode 340 into the assembly, but must allow sufficient exposure of an outer surface of the electrode 340 for contact with the user’s skin, when in use. According to some non-limiting aspects, the electrode may have a width “a” of between 15 and 35 milimeters. For example, the electrode may have a width “a” of 25 milimeters. The trace 335 contact may have a geometry similar to that of the electrode (e.g., circular), but may have a width smaller than that of the width “a” of the electrode, to ensure optimal electrical communication between the electrode 340 and the trace 335 contact. According to some non-limiting aspects, the trace 335 contact may have a width of between 10 and 30 milimeters. For example, the trace 335 contact may have a width of 20 milimeters. As such, it shall be appreciated that the width “a”, distance “b,” and overlap “d” are particularly configured such that the electrode 340 (or other sensor) is securely integrated within the assembly, while providing sufficient electrical contact between the electrode 340 and the trace 335 contact, and defining a sufficient aperture through which the outer surface of the electrode 340 can contact the skin of the user while in use.

An alternative trace deposition structure is depicted in FIG. 9B in cross section taken along lines A-A in FIG. 7. The structure is similar to that shown in FIG. 9A; however, the structure shown in FIG. 9B further includes a stencil layer 362 and a conductive layer 345 including conductive mesh, for example. More specifically, the stencil layer 362 is positioned adjacent the base substrate layer 360 for situations when the conductive traces 335 are not deposited directly onto the base substrate layer 360. Because the non-limiting aspect of 9B includes a conductive layer 345, the conductive layer 345 alleviates the need to consider a distance “b” inside of an outer edge, or boundary, of the electrode 340. However, as previously noted, the electrode 340 can include a first width “a” and the encapsulation layer 365 can be configured to overlap the electrode 340 by a distance “d.”

In some aspects, the traces 335 can be formed from a deformable conductor with specifically configured properties and/or the properties of the substrate 360 or layers surrounding the patterns of the deformable conductive material may be adjusted and/or optimized to ensure that the patterns of deformable conductive material heal upon unitization of the surrounding layers. For example, the deformable conductive material may be optimized to have a viscosity such that the deformable conductive material is able to heal upon unitization of the layers but not such that the deformable conductive material overly deforms and does not achieve the intended pattern. As another example, an adhesive characteristic and/or viscosity of the deformable conductive material may be optimized such that it remains on the substrate layer upon removal of the removable stencil and but does not adhere to the channels of the stencil thereby lifting the deformable conductive material off of the substrate layer. In some aspects, a viscosity of the deformable conductive material may, when under high shear (e.g., in motion), be in a range of about 10 Pascal seconds (Pa*s) and 500 Pa*s, such as a range of 50 Pa*s and 300 Pa*s, and/or may be about 50 Pa*s, about 60 Pa*s, about 70 Pa*s, about 80 Pa*s, about 90 Pa*s, about 100 Pa*s, about 110 Pa*s, about 120 Pa*s, about 130 Pa*s, about 140 Pa*s, about 150 Pa*s, about 160 Pa*s, about 170 Pa*s, about 180 Pa*s, about 190 Pa*s, or about 200 Pa*s. In some aspects, a viscosity of the deformable conductive material may, when under low shear (e.g., at rest), be in a range of 1,000,000 Pa*s and 40,000,000 Pa*s and/or may be about 10,000,000 Pa*s, about 20,000,000 Pa*s, about 30,000,000 Pa*s, or about 40,000,000 Pa*s.

Similarly, the traces 335 can include a deformable, conductive material, such as those disclosed in International Patent Application No. PCT/US2017/019762 titled LIQUID WIRE, which was filed on February 27, 2017 and published on September 8, 2017 as International Patent Publication No. WO2017/151523A1, the disclosure of which is hereby incorporated by reference in its entirety. For example, each trace 335 can include a variety of forms, such as a liquid, a paste, a gel, and/or a powder, amongst others that would enable the traces 335 to have a deformable (e.g., soft, flexible, stretchable, bendable, elastic, flowable viscoelastic, Newtonian, non-Newtonian, etc.) quality. According to some non-limiting aspects, the deformable, conductive materials can include an electroactive material, such as a deformable conductors produced from a conductive gel (e.g., a gallium indium alloy-based gel). The conductive gel can have a shear thinning composition and, according to some non-limiting aspects, can include a mixture of materials in a desired ratio. For example, according to one preferable non-limiting aspect, the conductive gel can include a weight percentage of a eutectic gallium alloy between 59.9% and 99.9% and a weight percentage of a gallium oxide between 0.1% and about 2.0%. Of course, the present disclosure contemplates other non-limiting aspects, featuring traces 335 of varying forms and/or compositions to achieve the benefits disclosed herein.

In some aspects, a viscosity of the deformable conductive material may, when under high shear (e.g., in motion), be in a range of about 10 Pascal seconds (Pa*s) and 500 Pa*s, such as a range of 50 Pa*s and 300 Pa*s, and/or may be about 50 Pa*s, about 60 Pa*s, about 70 Pa*s, about 80 Pa*s, about 90 Pa*s, about 100 Pa*s, about 110 Pa*s, about 120 Pa*s, about 130 Pa*s, about 140 Pa*s, about 150 Pa*s, about 160 Pa*s, about 170 Pa*s, about 180 Pa*s, about 190 Pa*s, or about 200 Pa*s. In some aspects, a viscosity of the deformable conductive material may, when under low shear (e.g., at rest), be in a range of 100,000 Pa*s and 40,000,000 Pa*s, such as a range of 1,000,000 Pa*s and 40,000,000 Pa*s, and/or may be about 10,000,000 Pa*s, about 20,000,000 Pa*s, about 30,000,000 Pa*s, or about 40,000,000 Pa*s.

In various instances, the traces 335 can terminate at and electrically couple to one or more electronic components 345 (e.g. processor, memory, transceiver, logic-based controller, pump, power source, light emitting diodes (“LEDs”), transducers, haptic sensors, connectors, contacts, etc.) Such electronic components can be mechanically secured to and/or otherwise integrated with the substrate 360 and/or electrically coupled to the traces 335 of the circuit. The electronic components can be configured to receive and utilize signals from the sensor array and electrical parameters from the traces 335 in accordance with the intended application. The electrical parameters may vary with a physical change to the circuit, for example an application of an external stimulus and/or deformation of the circuit. The resulting changes to the electrical parameters and the signals from the sensor array may be monitored, transmitted, or otherwise utilized to dynamically or statically calculate, infer or otherwise determine one or more attributes of the monitored body part.

Alternately and/or additionally, either a receiver, a transmitter, or a transceiver can be coupled to the substrate 360 and/or the sensor island, or patch, 330 and configured to transmit and/or receive signals to and/or from the sensor array 340, the puck 330, and/or its various electronic components. Alternately and/or additionally, feedback can be communicated to a user and/or wearer of the article. Such feedback can be audio, visual, and/or haptic in nature. For example, an LED array can be coupled to the substrate 360 and/or the sensor island 330 and configured to illuminate one or more LEDs based on electrical parameters (e.g., an inductance, a resistance, a voltage drop, a capacitance, signal, an electromagnetic field, etc.) generated by the traces 335 and/or structural parameters (e.g., a strain, a stress, a pressure, a dimension, etc.) determined by a microprocessor. Feedback may alternately or additionally be conveyed to a wearer through a display, e.g., OLED or LCD coupled to the substrate and/or the puck. Accordingly, various electronic components associated with such feedback communication can be coupled to the substrate 360 and/or the patch 330 and configured to monitor, transmit, and/or otherwise dynamically or statically calculate, infer or determine one or more physical or structural characteristics or conditions of the sensor array 340, the traces 335, the puck 330, and/or the monitored body part.

The sensor array 340 can include one or more electrodes that can be coupled to the stretchable substrate 360 in any suitable manner. In various instances, the one or more electrodes are button electrodes, which can be selectively snapped onto the substrate 360. The use of button electrodes provides a modularity component to the wearable article 100. For example, a user can couple any desired number of electrodes to the substrate 360 in any desired location. Such an ability to customize the number and/or position of such electrodes allows for the wearable article to be modular and increasingly effective in monitoring multiple body parts, for example. Furthermore, the components of the sensor array 340, such as the button electrodes, can be disposable and configured to be thrown away after each use. In other instances, the sensor array can be permanently affixed to the substrate 360 and intended for continued, multiple uses.

The detecting components of the sensor array can have any desirable form factor. For example, where the sensor array comprises electrodes, all of the electrodes can be round in shape. Stated another way, all of the sensing components can have the same geometry and dimension. The electrodes can have any geometry and dimensions suitable to provide the desired surface area for producing contact with the wearer’s body sufficient to monitor a particular body part. Such geometry includes generally square, rectangular, ovular, or irregular shapes. A combination of at least two different electrode shapes and/or sizes can be used in the sensor array to achieve an optimal amount of exposed sensing material adjacent to the target body part.

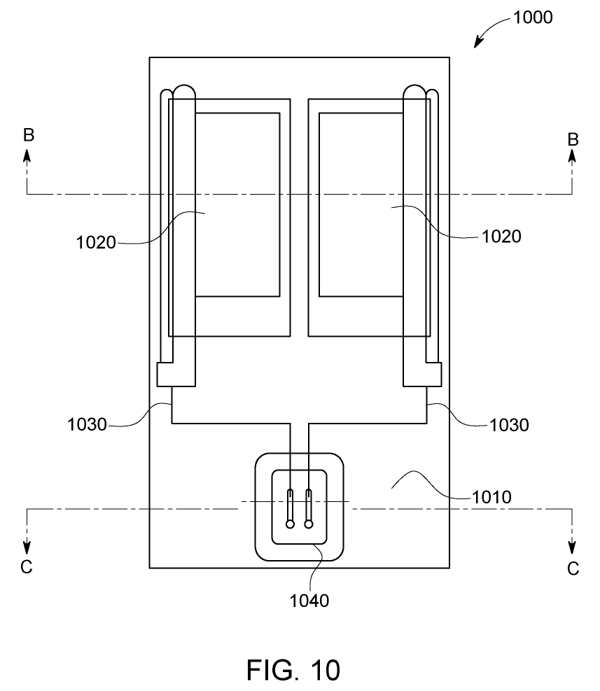

Referring now to FIG. 10, another sensor 1000 configured for use in any of the devices with flexible circuits disclosed herein is depicted according to at least one non-limiting aspect of the present disclosure. According to the alternate aspect of FIG. 10, the sensor 1000 can be configured as a sensor island, or patch. The patch 1000, similar in many aspects to the patch or sensor island 330 of FIG. 6, can include a stretchable substrate 1010, a pair of electrodes 1020, and an output device1040. The pair of electrodes 1020 are electrically coupled to the puck/output 1040 by traces 1030. In contrast to the electrodes 340, the electrodes 1020 are substantially rectangular in shape; however, any suitable shape is envisioned. Furthermore, the form factor of the patch 1000 is different than the rectangular form factor of the patch 330. The patch 1000 is able to be sized and/or shaped to effectively monitor a particular body part while comfortably fitting within the wearable article. For example, the patch 1000 can be configured for attachment to an inner surface of a garment, such as the inner surface 1502 of a sleeve 1500, as shown in FIG. 12. The patch 1000 is configured to be selectively positioned such that at least a portion of the pair of electrode’s exposed surface 1022 is in contact with the wearer’s body. For example, the patch 1000 can be positioned in a sleeve portion of a shirt, a torso portion of a shirt, or any other tubular, flat, or other shaped apparel structure and/or any other suitable location within any wearable article.

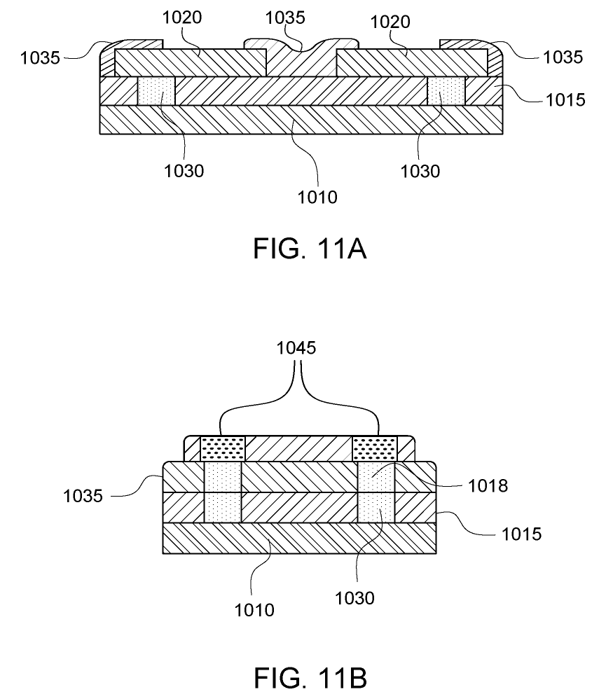

Referring now to FIGS. 11A and 11B, several cross-sections taken along lines B-B and C-C of FIG. 10, respectively, are depicted in accordance with at least one non-limiting aspect of the present disclosure. As previously discussed herein with respect to FIGS. 9A and 9B, various encapsulation techniques can be employed to maximize a skin-facing surface of the sensor array. Similarly, as shown in FIG. 11A, the patch 1000 is formed by various layers including a base substrate layer 1010, an optional stencil layer 1015, and a sensor 1020, such as an electrode. The optional stencil layer 1015 comprises gel traces 1030 deposited therein that are positioned to be in electrical contact with the sensor 1020. The sensor 1020 is encapsulated within the patch 1000 by an encapsulation layer 1035 that maintains the sensor 1020 in a desired orientation by overlapping at least a portion of the sensor 1020. It shall be appreciated that dimensioning similar to that employed in FIGS. 9A and 9B can be similarly applied in the non-limiting aspect of FIG. 11A, to ensure sufficient overlap, a secure integration of the sensor 1020, electrical contact between the sensor 1020 and the traces 1030, and sufficient contact between the sensor 1020 and the user’s skin. Such an overlapping relationship maintains the sensor 1020 in position while also protecting the electrical components from exposure to contaminants, such as a user’s sweat and/or water, for example. It shall be appreciated that the sensor 1020 can be integrated such that the encapsulation layer 1035 has a predetermined thickness that recesses the outer surface of the sensor 1020 without preventing sufficient contact with the skin of the user. However, according to other non-limiting aspects, the sensor 1020 can include any of the sensors disclosed in International Patent Application No. PCT/US2022/071012, titled DEVICES, SYSTEMS, AND METHODS TO MONITOR AND CHARACTERIZE THE MOTIONS OF AUSER VIA FLEXIBLE CIRCUITS, and filed March 7, 2022, the disclosure of which is herein incorporated by reference in its entirety. Accordingly, according to some non-limiting aspects, the sensors 1020 can have a curved or domed surface that is not recessed, but at least partially protrudes beyond the encapsulation layer 1035 such that the sensor 1020 can be biased against the skin of the user in use. The encapsulation layer 1035 further comprises contacts 1018, or vias, configured like a traditional printed circuit board (“PCB”), e.g., acting as the pathways for signals, currents or potentials to travel between the output(s) 1045 shown in FIG. 11B and other auxiliary structures, e.g., sensors. Outputs 1045 may be copper contacts encapsulated in polyimide, e.g., a flex PCB. For simplicity and clarity, some layers have been omitted from the cross section view. The outputs 1045 shown surrounded by polyimide in this embodiment in practice would be encapsulated at the top and bottom with polyimide film, and vias on the top would permit connections to be made to an external device or electronic component, and vias at the bottom would permit electrical communication between outputs 1045 and vias 1018. As previously described in reference to FIG. 11A, the traces 1030 can be configured to extend across the entirety or even beyond the limits of the sensor 1020, which can be an electrode. Whereas FIGS. 9A and 9B illustrate a circular configuration of traces 335 for the electrical interface, defining a diameter that is smaller than a diameter of the applied sensors 340, the electrical interface of FIGS. 11A and 11B illustrates a different approach, wherein the traces terminate in a contact pad with larger dimensions than the sensor 1020. The configuration of FIGS. 11A and 11B, for example, may provide a more robust electrical interface between the sensor 1020 and traces 1030. According to some non-limiting aspects, another layer, such as a polyimide layer, can be further disposed over the output(s) 1045, as desired.

In various instances, the microprocessor has a memory having stored instructions. When executed, the instructions allow the microprocessor to analyze and make determinations based on the signals detected and communicated by the sensor array.

As discussed in greater detail herein, the wearable article is configured to have one or more pairs of electrodes and/or discrete sensors (e.g., IMU’s, strain sensors, pressure sensors, temperature sensors, etc.) positioned thereon. The microprocessor is configured to recognize and determine which, if any, one or more electrodes, pairs of electrodes, and/or other sensor(s) are detecting meaningful data. For example, when the electrodes are EMG electrodes, only the electrodes that have a maintained contact with a user’s skin are detecting meaningful data. One or more electrodes may be out of contact with a user’s skin for various reasons. For example, the wearable article may not be secured tightly enough with respect to the user. In other instances, the wearable article may be worn incorrectly. Portions of the wearable article may be overlapped with one another as discussed with respect to the wrap 100 shown in FIGS. 1-5. In such instances, one or more electrodes may not be in contact with the user’s skin. Instead, a layer of the wearable article may be positioned in between the one or more electrodes and the user’s skin thereby impeding the detection of any valuable signals. The processor is configured to identify such ineffective one or more electrodes by assessing the strength of the signal(s) received therefrom. For example, the processor can filter out the signals emitted by the ineffective electrodes based on a signal with a strength below a predetermined threshold value. The processor can further or alternatively filter out such signals by comparing their strength(s) to that of other signals received by other electrodes on the wearable article. In instances where the processor determines one or more signal strengths are below a desired threshold and/or are different than other signal strengths, the processor can ignore such signals and/or deactivate the electrodes by causing a power supply to stop providing the electrodes with power, for example. According to some non-limiting aspects, ignoring can include not storing or logging sampled signal values. According to other non-limiting aspects, ignoring can include turning off a channel associated with a sensor that is generating the value that doesn’t meet the signal criteria.

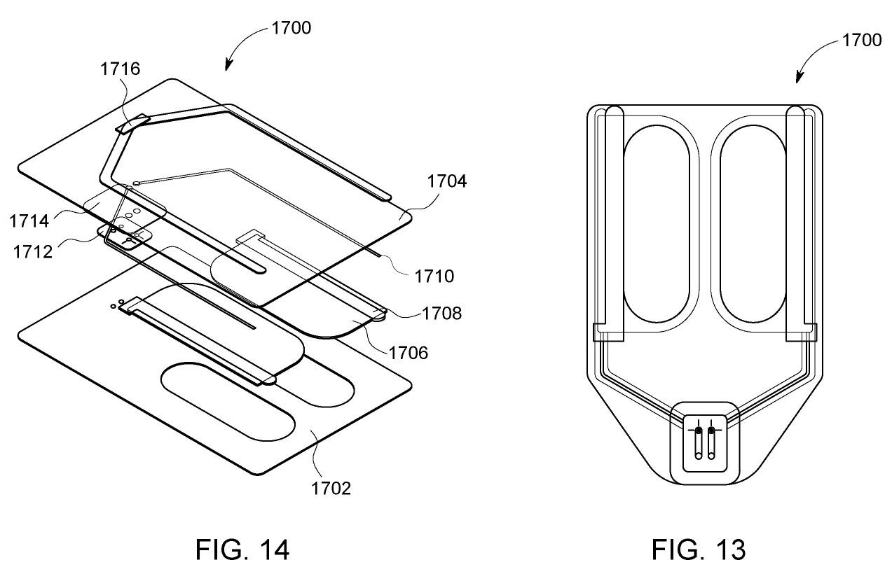

Referring now to FIG. 13, another sensor 1700 configured for use in any of the devices with flexible circuits disclosed herein is depicted according to at least one non-limiting aspect of the present disclosure. Likewise, FIG. 14 illustrates an exploded, perspective view of the sensor 1700 of FIG. 13, illustrating how the sensor 1700 can include a via layer 1702, a fabric layer 1704, electrodes 1706, barriers 1708, an output 1712, which can take the form of a sensor island or a puck in some non-limiting aspects, traces 1710 formed from deformable conductors, a trapping layer 1714 to cover and encapsulate the output 1712, and encapsulation layer(s) 1716 for encapsulating the traces 1710. The fabric layer 1704 and the via layer 1702 may form the external surfaces of the patch, and the remaining components are interposed between them, and the entire assembly may be unitized using at least one of heat and pressure as previously discussed for similar structures. Specifically, the via layer 1702 can be configured to interface with the skin of a user when the device is worn and thus, may include two cutouts, predefined to allow a certain contact area of the electrodes 1706 contact the skin when the sensor 1700 is in use. The electrodes 1706 in this example are formed from a conductive mesh fabric, such as one woven from Shieldex™ conductive yarns. Thus, the barriers 1708 are provided to prevent unwanted migration of the deformable traces 1710 outside of the mesh material of the electrodes 1706 and come into contact with other components of the sensor 1700, the wearer, and/or device. The barriers 1708 may be diffused within the electrode and act as the substrate layer for the traces 1710, thereby encapsulating them in the regions of the electrode, whereas the trace layer 1702 and the encapsulating layer(s) 1716 may serve to encapsulate the traces in the regions away from the electrodes.

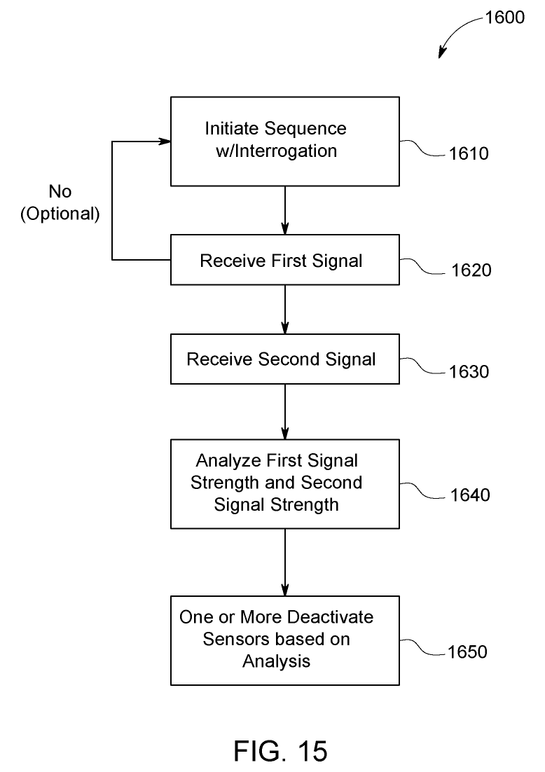

Referring now to FIG. 15, a method 1600 of selectively monitoring a body part of a user by comparing sensor signals generated by flexible circuits of a wearable article is depicted in accordance with at least one non-limiting aspect of the present disclosure. According to the non-limiting aspects of FIG. 15, the method 1600 can include initiating 1610 an interrogation of a sensor array of a wearable article via a user interface of an application executed or otherwise accessed by a processor or other computing device. The method 1600 can then include receiving a first signal 1620 from a first sensor of the sensor array, wherein the first signal has a first signal strength. The method 1600 can further include receiving a second signal 1630 from a second sensor of the sensor array, wherein the second signal has a second signal strength. The method 1600 can further include analyzing 1640 the first signal strength and the second signal strength. Such an analysis 1640 can include comparing the first signal strength and the second signal strength. Such an analysis 1640 can include comparing the received signal strengths against a predetermined threshold. The method 1600 can further include causing one or more sensors to be deactivated 1650 based on the analysis of the signal strengths. The method can involve deactivating 1650 one or more sensors for various reasons, including, for example, the first sensor strength falls below a pre-determined threshold indicative of the first sensor not being in sufficient contact with a user’s skin, the first sensor strength is less than the second signal strength indicative of the second sensor being in a more desirable contact with the user’s skin, and/or the failure of the processor to receive a first signal from the first sensor. In such instances, the processor can deactivate 1650 the first sensor by preventing a power source from providing power to the first signal thereby turning the first sensor off. In various instances, the processor can deactivate 1650 the first sensor by ignoring the signal emitted by the first sensor until a threshold is reached. In such instances, the processor can continue to perform an interrogation loop until a suitable signal 1620 is received from the first sensor.

In various instances, the memory stores threshold values to facilitate the microprocessor’s assessment of the detected electrode signal. In instances where a signal is unable to be detected and/or the signal strength is below a stored baseline threshold, the microprocessor is configured to turn off and/or ignore any future signals coming from the particular electrode. In other instances, the microprocessor is configured to ignore any signals from the particular electrode until a signal is received that exceeds the baseline threshold.

The memory of the microprocessor may comprise instructions to permit the microprocessor to determine the particular portion of the user’s body that is in contact with the wearable article. For example, the memory may comprise a stored set of values and/or thresholds that are unique to a particular body portion. After receiving the signals detected by the sensor array, the microprocessor is configured to analyze the detected signals against the stored set of values and/or thresholds to identify the monitored body portion.

Upon identifying the particular body portion being monitored by the wearable article, the microprocessor is further configured to focus on a select portion of the sensor array. Stated another way, the microprocessor is configured to selectively analyze the signals detected by a particular grouping of sensors, such as electrodes, on the wearable article based on the body part(s) adjacent to the wearable article. In such instances, the microprocessor can deactivate one or more electrodes while the wearable article is adjacent a specific body part.

The stretchable substrate may be melted onto and/or infused directly onto or into the fabric of the wearable article; however, any suitable attachment mechanism to securely affix the substrate to the wearable article is envisioned. In some embodiments, a textile adhesive such as Bemis® 3292 or Bemis ® EverFit™ 6430 may be used to adhere a stretchable substrate including the flexible circuitry and sensing unit to the wearable article. Or, the wearable article may be manufactured in such a way that the stretchable substrate is integrated within a panel or portion of the wearable garment, such as between an exterior and an interior layer of the garment. Given the nature and intended uses of the disclosed wearable articles, exposure of the stretchable substrate to water and/or other bodily fluids, such as sweat, are expected. The disclosed wearable articles are intended to be reused. As such, the wearable articles contain washable circuit architectures. Care must be taken to prevent liquid from contaminating the circuitry components. Methods of forming a migratory barrier to achieve a hermetic and/or fluid seal are disclosed in greater detail in International Patent Application Serial No. PCT/US2022/072371, titled FLEXIBLE HIGH-POWER ELECTRONICS BUS, filed on May 17, 2022, and U.S. Provisional Patent Application Serial Nos. 63/201,902, filed May 18, 2021 and 63/201,915, filed May 18, 2021, the disclosures of which are each incorporated by reference herein in their entireties.

Examples of the method according to various aspects of the present disclosure are provided below in the following numbered clauses. An aspect of the method may include any one or more than one, and any combination of, the numbered clauses described below.

Clause 1: A monitoring system, including a flexible wrap configured to be secured to a user, the flexible wrap including a layer of material, and a stretchable substrate coupled to the layer of material, wherein the stretchable substrate includes a circuit including an encapsulated deformable conductor and a sensor array, a processor communicably coupled to the circuit, and a memory configured to store instructions that, when executed by the processor, cause the processor to receive a signal from the sensor array, compare a strength of the received signal to a stored baseline threshold value, and ignore the received signal based on the signal strength being less than the baseline threshold value.

Clause 2: The monitoring system according to Clause 2, wherein the stretchable substrate further includes a power source attached thereto.

Clause 3: The monitoring system according to either of Clauses 1 or 2, wherein the encapsulated deformable conductor includes a pattern of traces.

Clause 4: The monitoring system according to any of Clauses 1-3, wherein the pattern of traces is configured to change its viscosity in response to a strain applied to the stretchable substrate.

Clause 5: The monitoring system according to any of Clauses 1-4, wherein the sensor array includes an electromyography electrode.

Clause 6: The monitoring system according to any of Clauses 1-5, wherein the stretchable substrate further includes an inertial measurement unit coupled thereto.

Clause 7: The monitoring system according to any of Clauses 1-6, wherein the processor is configured to deactivate at least a portion of the sensor array based on the received signal being less than the baseline threshold value.

Clause 8: The monitoring system according to any of Clauses 1-7, wherein the processor is configured to deactivate at least a portion of the sensor array based on the processor failing to receive the signal from the sensor array.

Clause 9: A wearable article, including a stretchable fabric, a flexible circuit coupled to the stretchable fabric, wherein the flexible circuit includes a first electrode including an outer surface of a first material and an inner surface of a second material, a substrate layer, a trace formed from a deformable conductor and electrically coupled to the second material of the first electrode, and an encapsulation layer, wherein the encapsulation layer is configured to completely cover the trace and partially cover the first surface of the first electrode, a processor communicably coupled to the circuit, and a memory configured to store instructions that, when executed by the processor, cause the processor to receive a first signal from the first electrode, receive a second signal from a second electrode, compare a first strength of the received first signal to a second strength of the received second signal, and deactivate the first electrode based on the comparison of the first strength and the second strength.

Clause 10: The wearable article according to Clause 9, wherein the first sensor is an electrode.

Clause 11: The wearable article according to either of Clauses 9 or 10, wherein the first material includes a doped silicone.

Clause 12: The wearable article according to any of Clauses 9-11, wherein the encapsulated deformable conductor includes a pattern of traces.

Clause 13: The wearable article according to any of Clauses 9-12, wherein the pattern of traces are printed on the stretchable substrate.

Clause 14: The wearable article according to any of Clauses 9-13, wherein the pattern of traces is configured to serve as a strain sensor.

Clause 15: The wearable article according to any of Clauses 9-14, wherein the processor is configured to deactivate the first sensor by causing a power source to discontinue supplying power to the first sensor.

Clause 16: The wearable article according to any of Clauses 9-15, wherein the processor is configured to deactivate the first sensor based on the first signal strength being less than the second signal strength.

Clause 17: The wearable article according to any of Clauses 9-16, wherein the processor is configured to deactivate the first sensor based on the processor failing to receive the first signal from the first sensor.

Clause 18: The wearable article according to any of Clauses 9-17, wherein the stretchable substrate is infused into the layer of fabric.

Clause 19: The wearable article according to any of Clauses 9-18, wherein the sensor is positioned adjacent to a tissue of a user.

Clause 20: The wearable article according to any of Clauses 9-19, wherein the encapsulation layer partially covers at least one of the first sensor or the second sensor.

Clause 21: The wearable article according to any of Clauses 9-20, wherein the trace is electrically coupled to the first sensor via an electrical contact including a predetermined aspect ratio.

Clause 22: The wearable article according to any of Clauses 9-21, wherein the predetermined aspect ratio is greater than or equal to X and less than or equal to Y.

Clause 23: The wearable article according to any of Clauses 9-22, wherein the predetermined aspect ratio is Z.

Clause 24: The wearable article according to any of Clauses 9-23, wherein an outer surface of the first sensor includes a doped silicone, and wherein an inner surface of the first sensor includes a second material, wherein the first material is different than the second material.

Clause 25: The wearable article according to any of Clauses 9-24, wherein the first material includes a doped silicone.

Clause 26: The wearable article according to any of Clauses 9-25, wherein the second material includes a conductive textile.

Clause 27: A computer-implemented method of selectively monitoring a body part of a user via a wearable article including a stretchable substrate coupled to a layer of fabric, wherein the stretchable substrate includes a circuit including a deformable encapsulated conductor, a first sensor, and a second sensor, the method including receiving, via a processor, a first signal from the first sensor, wherein the first signal includes a first signal strength, receiving, via the processor, a second signal from the second sensor, wherein the second signal includes a second signal strength, comparing, via the processor, the first signal strength to the second signal strength, and causing, via the processor, the first sensor to be deactivated based on said comparison of the first signal strength to the second signal strength.

Clause 28: The devices disclosed herein.

Clause 29: The systems disclosed herein.

Clause 30: The methods disclosed herein.

All patents, patent applications, publications, or other disclosure material mentioned herein, are hereby incorporated by reference in their entirety as if each individual reference was expressly incorporated by reference respectively. All references, and any material, or portion thereof, that are said to be incorporated by reference herein are incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference and the disclosure expressly set forth in the present application controls.

The present invention has been described with reference to various exemplary and illustrative aspects. The aspects described herein are understood as providing illustrative features of varying detail of various aspects of the disclosed invention; and therefore, unless otherwise specified, it is to be understood that, to the extent possible, one or more features, elements, components, constituents, ingredients, structures, modules, and/or aspects of the disclosed aspects may be combined, separated, interchanged, and/or rearranged with or relative to one or more other features, elements, components, constituents, ingredients, structures, modules, and/or aspects of the disclosed aspects without departing from the scope of the disclosed invention. Accordingly, it will be recognized by persons having ordinary skill in the art that various substitutions, modifications or combinations of any of the exemplary aspects may be made without departing from the scope of the invention. In addition, persons skilled in the art will recognize, or be able to ascertain using no more than routine experimentation, many equivalents to the various aspects of the invention described herein upon review of this specification. Thus, the invention is not limited by the description of the various aspects, but rather by the claims.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although claim recitations are presented in a sequence(s), it should be understood that the various operations may be performed in other orders than those which are described, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

Directional phrases used herein, such as, for example and without limitation, top, bottom, left, right, lower, upper, front, back, and variations thereof, shall relate to the orientation of the elements shown in the accompanying drawing and are not limiting upon the claims unless otherwise expressly stated.

The terms “about” or “approximately” as used in the present disclosure, unless otherwise specified, means an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 200%, 105%, 100%, 9%, 8%, 7%, 6%, 5%, 4%, 3%, 2%, 1%, 0.5%, or 0.05% of a given value or range.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Any numerical range recited herein includes all sub-ranges subsumed within the recited range. For example, a range of “1 to 100” includes all sub-ranges between (and including) the recited minimum value of 1 and the recited maximum value of 100, that is, having a minimum value equal to or greater than 1 and a maximum value equal to or less than 100. Also, all ranges recited herein are inclusive of the end points of the recited ranges. For example, a range of “1 to 100” includes the end points 1 and 100. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, any reference to a processor or microprocessor can be substituted for any “control circuit,” which may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system’s registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

What is claimed is:

1. A monitoring system, comprising:
an article configured to be worn by a user, the article comprising:
a layer of material; and
a substrate coupled to the layer of material, wherein the substrate comprises a circuit comprising an encapsulated deformable conductor and a sensor array;
a processor communicably coupled to the circuit; and
a memory configured to store instructions that, when executed by the processor, cause the processor to:
receive a signal from the sensor array;
compare a parameter of the received signal to at least one stored signal criteria; and
ignore the received signal if it does not meet the stored signal criteria.

2. The monitoring system of claim 1, wherein the substrate further comprises a power source coupled thereto.

3. The monitoring system of claim 1, wherein the encapsulated deformable conductor comprises a pattern of traces.

4. The monitoring system of claim 3, wherein the pattern of traces is configured to change its viscosity in response to a strain applied to the substrate.

5. The monitoring system of claim 1, wherein the sensor array comprises at least one pair of electromyography electrodes.

6. The monitoring system of claim 1, wherein the substrate further comprises an inertial measurement unit coupled thereto.

7. The monitoring system of claim 1, wherein the sensor array comprises at least one sensor.

8. The monitoring system of claim 1, wherein the sensor array comprises at least two sensors.

9. The monitoring system of claim 8, wherein the instruction to ignore the signal causes the processor to deactivate one of the at least two sensors based on the received signal from that one sensor not meeting the at least one stored sensor criteria.

10. The monitoring system of claim 1, wherein the processor is configured to deactivate at least a portion of the sensor array based on the processor failing to receive the signal a sensor in the array.

11. The monitoring system of claim 1 wherein the substrate is stretchable.

12. The monitoring system of claim 1, wherein the article comprises a flexible wrap.

13. A computer-implemented method of selectively monitoring a body part of a user via a wearable article comprising a substrate coupled to a layer of fabric, wherein the substrate comprises a circuit comprising a deformable encapsulated conductor, a first sensor, and a second sensor, the method comprising:
receiving, via a processor, a first signal from the first sensor, wherein the first signal comprises a first signal strength;
receiving, via the processor, a second signal from the second sensor, wherein the second signal comprises a second signal strength;
comparing, via the processor, the first signal and a second signal; and
causing, via the processor, the first sensor to be deactivated based on the comparison of the first signal and second signal.

14. The computer-implemented method of claim 13 wherein the first sensor comprises a first pair of electrodes and the second sensor comprises a second pair of electrodes.

15. A wearable article, comprising:
a fabric;
a flexible circuit coupled to the fabric, wherein the flexible circuit comprises:
a first pair of electrodes comprising a first electrode and a second electrode;
a second pair of electrodes comprising a third electrode;
each of the first, second, and third electrodes having an inner surface and an outer surface;
a substrate layer;
a trace formed from a deformable conductor and electrically coupled to the inner surface of each electrode; and
an encapsulation layer, wherein the encapsulation layer is configured to completely cover the trace and partially cover the outer surface of each electrode;
a processor communicably coupled to the circuit; and
a memory configured to store instructions that, when executed by the processor, cause the processor to:
receive a first signal from the first pair of electrodes;
receive a second signal from the second pair of electrodes;
compare the first signal and the second signal; and
deactivate one of the first and second electrode pairs based on the comparison of the first and second signals.

16. The wearable article of claim 15, wherein the second pair of electrodes comprises a fourth electrode.

17. The wearable article of claim 15, wherein at least one of the electrodes comprises a doped silicone material.

18. The wearable article of claim 15, wherein the deformable conductor comprises a pattern of traces.

19. The wearable article of claim 18, wherein the pattern of traces are deposited on the substrate.

20. The wearable article of claim 19, wherein at least a portion of the pattern of traces is configured to serve as a strain sensor.
